(19) United States
(12) Patent Application Publication
(10) Pub. No.: US 2023/0107484 A1
(43) Pub. Date: Apr. 6, 2023

(54) REDUCTION OF NEUTROPHIL EXTRACELLULAR TRAP FORMATION BY MESENCHYMAL STEM CELLS AND THEIR EXOSOMES

(71) Applicant: Therapeutic Solutions International, Inc., Oceanside, CA (US)

(72) Inventors: Thomas E. Ichim, Oceanside, CA (US); Famela Ramos, Oceanside, CA (US); James Veltmeyer, Oceanside, CA (US); Timothy G. Dixon, Oceanside, CA (US); Feng Lin, Oceanside, CA (US); Wais Kaihani, Oceanside, CA (US)

(73) Assignee: Therapeutic Solutions International, Inc., Oceanside, CA (US)

(21) Appl. No.: 17/960,047

(22) Filed: Oct. 4, 2022

Related U.S. Application Data
(60) Provisional application No. 63/251,993, filed on Oct. 4, 2021.

(51) Int. Cl. A61K 35/28; C12N 5/071

(52) U.S. Cl. CPC A61K 35/28; C12N 5/0602

(57) ABSTRACT

Disclosed are methods of reducing lung inflammation in acute respiratory distress syndrome elicited by various factors such as COVID-19 infection by reduction of neutrophil extracellular trap formation through administration of mesenchymal stem cells and/or exosomes thereof. The invention provides means of inhibiting neutrophil release of extracellular traps by mesenchymal stem cells and/or exosomes derived from said mesenchymal stem cells. Additionally, synergies are provided between mesenchymal stem cells and/or exosomes derived from mesenchymal stem cells and agents approaches which reduce neutrophil extracellular trap formation.

### CROSS-REFERENCE TO RELATED APPLICATIONS

This application claims priority to U.S. Provisional Application Ser. No. 63/251,993, titled “Reduction of Neutrophil Extracellular Trap formation by Mesenchymal Stem Cells and their Exosomes”, filed Oct. 4, 2021, which is hereby incorporated by reference in its entirety.

### FIELD OF THE INVENTION

The invention herein relates to the treatment of COVID-19, and more particularly modifying the immune system by reducing neutrophil extracellular trap formation.

### BACKGROUND

In 2004 Brinkmann et al published a bizarre observation: that upon specific activation, neutrophils release granule proteins and chromatin that together form extracellular fibers that bind Gram-positive and -negative bacteria. They termed these strange fibers neutrophil extracellular traps (NETs). The researchers found that NETs degrade virulence factors and kill bacteria. Furthermore, they observed that NETs are abundant in vivo in experimental dysentery and spontaneous human appendicitis, two examples of acute inflammation. In the paper the researchers concluded that NETs appear to be a form of innate response that binds microorganisms, prevents them from spreading, and ensures a high local concentration of antimicrobial agents to degrade virulence factors and kill bacteria [1].

It is known that neutrophils, in part due to their differentiated nature and relatively short half-life, are primarily transcriptionally inactive and their DNA is condensed into heterochromatin within the nucleus [2-7]. As with other cells, in the neutrophil nucleosome is composed of DNA is wrapped around histones. The nucleosomes are further organized into chromatin. For the heterochromatin to open, that is, “decondense” then enzyme peptidyl arginine deiminase 4 (PAD4) is critical because it catalyzes the conversion of histone arginines to citrullines, thus reducing the strong positive charge of histones and consequently weakening histone-DNA binding [8-18]. This reduced interaction permits for the unravelling of the nucleosomes, a which is essential for NET formation. Rapid increases in intracellular Ca2+ are needed for intracellular signal transduction during normal neutrophil activation [19-30].

### SUMMARY

Preferred embodiments are directed to methods of reducing neutrophil secretion of neutrophil extracellular traps comprising administering a mesenchymal stem cell and/or products derived from said mesenchymal stem cells to a patient in need of treatment.

Preferred methods include embodiments wherein said mesenchymal stem cells are activated in a manner to enhance ability to inhibit neutrophil production of NETs by direct contact or by secretion of soluble factors.

Preferred methods include embodiments, wherein said NETs are comprised of DNA and histones.

Preferred methods include embodiments wherein said mesenchymal stem cells are autologous to the host.

Preferred methods include embodiments wherein said mesenchymal stem cells are allogeneic to the host.

Preferred methods include embodiments wherein said mesenchymal stem cells are xenogeneic to the host.

Preferred methods include embodiments wherein said mesenchymal stem cells are derived from a tissue selected from a source selected from the group consisting of: a) bone marrow; b) liver; c) spleen; d) adipose tissue; e) peripheral blood; f) mobilized peripheral blood; g) cerebral spinal fluid; h) menstrual blood; i) tonsils; j) deciduous tooth; k) fallopian tube; l) endometrium; m) muscle; and n) hair follicle.

Preferred methods include embodiments wherein said mesenchymal stem cells express SCA-

Preferred methods include embodiments wherein said mesenchymal stem cells express interleukin-1 receptor.

Preferred methods include embodiments wherein said mesenchymal stem cells express interleukin-3 receptor.

Preferred methods include embodiments wherein said mesenchymal stem cells express interleukin-6 receptor.

Preferred methods include embodiments wherein said mesenchymal stem cells express interleukin-10 receptor.

Preferred methods include embodiments wherein said mesenchymal stem cells express leukemia inhibitor factor receptor.

Preferred methods include embodiments wherein said mesenchymal stem cells express HGF-1 receptor.

Preferred methods include embodiments wherein said mesenchymal stem cells express VEGF receptor.

Preferred methods include embodiments wherein said mesenchymal stem cells express CD133.

Preferred methods include embodiments wherein said me8senchymal stem cells express CD90.

Preferred methods include embodiments wherein said mesenchymal stem cells express PD-L1.

Preferred methods include embodiments wherein said mesenchymal stem cells express CTLA-4

Preferred methods include embodiments wherein said mesenchymal stem cells express FoxP3.

Preferred methods include embodiments, wherein said mesenchymal stem cells express PDGF-receptor.

Preferred methods include embodiments, wherein said mesenchymal stem cells express CD105.

Preferred methods include embodiments, wherein said mesenchymal stem cells express CD73.

Preferred methods include embodiments, wherein said mesenchymal stem cells express CD37.

Preferred methods include embodiments, wherein said mesenchymal stem cells express Galectin-3.

Preferred methods include embodiments, wherein said mesenchymal stem cells express Galectin-9.

Preferred methods include embodiments, wherein said mesenchymal stem cells express MMP-3.

Preferred methods include embodiments, wherein said mesenchymal stem cells express MMP-7.

Preferred methods include embodiments, wherein said mesenchymal stem cells express MMP-9.

Preferred methods include embodiments, wherein said mesenchymal stem cell is immune modulatory.

Preferred methods include embodiments, wherein said immunomodulatory activity is ability to suppress proliferation in a mixed lymphocyte reaction by more than 10% as compared to a fibroblast.

Preferred methods include embodiments, wherein said immunomodulatory activity is ability to suppress inflammatory cytokine production in a mixed lymphocyte reaction by more than 10% as compared to a fibroblast.

Preferred methods include embodiments, wherein said inflammatory cytokine is TNF-alpha.

### BRIEF DESCRIPTION OF THE DRAWINGS

FIG. 1 is a bar graph showing neutrophil extracellular trap formation being reduced by activated exosomes.

### DETAILED DESCRIPTION OF THE INVENTION

The invention provides means of reducing inflammation in the lungs of patients with acute respiratory distress syndrome (ARDS), and especially in patients with ARDS caused by COVID-19. It is known that COVID-19 pathology is caused in many cases by lung inflammation. There are numerous components that are in some cases causative and in some cases associated with the lung damage. The invention teaches that by suppressing neutrophil release of NETs as well as by inactivating NETs, we are able to reduce COVID-19 pathology.

NETs have been described in numerous conditions associated with COVID-19. For example, one of the first suggestions of NETs playing a lethal role in COVID-19 infection was an autopsy report published in the Journal of Translational Medicine describing histones and neutrophils that appeared to have released their NETs in the lungs of a patient who succumbed to disease [31].

In some embodiments, the invention teaches means of generating neonatal NETS inhibiting factor, or compounds similar to it, through the use of mesenchymal stem cells or other regenerative cells.

In some embodiments of the invention reduction of neutrophil extracellular traps accomplished by administration of MSC and/or MSC derivatives is utilized to reduce expansion of aneurysms such as aortic aneurysms [32].

In some embodiments of the invention inhibitors of complement are utilized together with exosomes from mesenchymal stem cells to inhibit pulmonary damage. Various inhibitors of complement activation are known and include Ravulizumab [33-40] and ecluzimab [33, 41-46].

In some embodiments of the invention, inhibition of NETs is performed in order to prevent autoimmunity such as type 1 diabetes. It has been demonstrated in the art that NETs may contribute to forcing of dendritic cell maturation, which is involved in the pathogenesis of this disease [47]. Other studies have reported dendritic cell maturation [48-50], as well as macrophage activation occur in response to contact with NETs [51, 52].

In some embodiments of the invention agents that inhibit NETs such as erythromycin [53], dimethylfumarate [54], are administered prior to, and/or concurrently with, and/or subsequent to administration of MSC and/or derivatives thereof.

In one embodiment of the invention MSC useful for suppression of NET production and/or NET activity are produced by cultivating the mesenchymal stem cell population in a culture medium comprising DMEM (Dulbecco's modified eagle medium), F12 (Ham's F12 Medium), M171 (Medium 171) and FBS (Fetal Bovine Serum. In some embodiments of the invention MSC and/or media conditioned by MSC possess enhanced ability to inhibit NET formation and/or inhibit NET activity by the expression and/or secretion of at least one, two, three or all four of Angiopoietin 1 (Ang-1), TGF-.beta.1, VEGF, and HGF by the MSC.

In this context, it is also noted that the present invention has the further surprising advantage that cultivation in the culture medium of the present invention provides for the isolation of a mesenchymal stem cell population such as an mesenchymal stem cell population of the amniotic membrane of umbilical cord of which more than 90%, or even 99% or more of the cells are positive for the three MSC CD73, CD90 and while at the same these stem cells lack expression of CD34, CD45 and HLA-DR, meaning 99% or even more cells of this population express the stem cell markers CD73, CD90 and CD105 while not expressing the markers CD34, CD45 and HLA-DR. Such an extremely homogenous and well-defined cell population is the ideal candidate for clinical trials and cell-based therapies since, they for example, fully meet the criteria generally accepted for human mesenchymal stem cells to be used for cellular therapy as defined, for example, by Dominici et al, “Minimal criteria for defining multipotent mesenchymal stromal cells. The International Society for Cellular Therapy position statement”, Cytotherapy (2006) Vol. 8, No. 4, 315-317, Sensebe et al., “Production of mesenchymal stromal/stem cells according to good manufacturing practices: a, review”, Stem Cell Research & Therapy 2013, 4:66), Vonk et al., Stem Cell Research & Therapy (2015) 6:94, or Kundrotas Acta Medica Lituanica. 2012. Vol. 19. No. 2. P. 75-79. Also, using a bioreactor such as a Quantum Cell Expansion System, it is possible to obtain high numbers of MSC such as 300 to 700 million mesenchymal stem cells per run. Thus, the present invention provides the further advantage to provide the amounts of stem cells that are needed for therapeutic applications such as their use in wound healing in a cost efficient manner. In addition, all components used for making the culture medium of the present invention are commercially available in GMP quality. Accordingly, the present invention opens the route to the GMP production of a highly homogenous MSC population, for example of placental tissue or umbilical cord tissue, for example, a mesenchymal stem cell population of the amniotic membrane of the umbilical cord or a mesenchymal stem cell population of Wharton's jelly. The invention contemplates obtaining MSC from other sources as well, including bone marrow [55-80], aldehyde dehydrogenase high bone marrow stem cells [59, 81-84], sca-1 positive bone marrow mesenchymal stem cells [85], peripheral blood, adipose [86], mobilized peripheral blood, menstrual blood, intraventricular fluid, cerebral spinal fluid, muscle, hair follicle, tonsils [87], nail follicle, deciduous tooth, lung [88], heart [89], and urine.

In some embodiments of the invention stem cells are pretreated with low dose hydrogen peroxide to augment ability to suppress NET formation and/or NET activity. Treatment of cells with low dose hydrogen peroxide is described in this publication and incorporated by reference [90].

In one embodiment, the MSC population utilized for reducing NET production and/or activity is derived from the umbilical cord from any compartment of umbilical cord tissue that contains mesenchymal stem cells. The MSC population may be a mesenchymal stem cell population of the amnion (AM), a perivascular (PV) mesenchymal stem cell population, a mesenchymal stem cell population of Wharton's jelly (WJ), a mesenchymal stem cell population of the amniotic membrane of umbilical cord but also a mixed mesenchymal stem cell population of the umbilical cord (MC), meaning a population of mesenchymal stem cells that includes stem cells of two or more of these compartments. Mesenchymal stem cells of these compartments and the isolation therefrom are known to the person skilled in the art and are described, for example, by Subramanian et al “Comparative Characterization of Cells from the Various Compartments of the Human Umbilical Cord Shows that the Wharton's Jelly Compartment Provides the Best Source of Clinically Utilizable Mesenchymal Stem Cells”, PLoS ONE 10(6): e0127992, 2015 and the references cited therein, Van Pham et al. “Isolation and proliferation of umbilical cord tissue derived mesenchymal stem cells for clinical applications”, Cell Tissue Bank (2016) 17:289-302, 2016. A mixed mesenchymal stem cell population of the umbilical cord can, for example, be obtained by removing the arteries and veins from the umbilical cord tissue, cutting the remaining tissue and the Wharton's jelly into piece and and cultivating the umbilical cord tissue (by tissue explant) in the culture medium of the present invention. A mixed mesenchymal stem cell population of the umbilical cord may also be obtained by culturing entire umbilical cord tissue with intact umbilical vessels as tissue explant under the conditions (cultivation in serum-supplemented DMEM with 10% fetal bovine serum, 10% horse serum, and 1% Penicillin/Streptomycin) as described by Schugar et al. “High harvest yield, high expansion, and phenotype stability of CD146 mesenchymal stromal cells from whole primitive human umbilical cord tissue. Journal of biomedicine & biotechnology. 2009; 2009:789526”. In this context, it is noted that a mesenchymal stem cell population of the cord-placenta junction can be isolated as described by Beeravolu et al. “Isolation and Characterization of Mesenchymal Stromal Cells from Human Umbilical Cord and Fetal Placenta.” J Vis Exp. 2017; (122): 55224.

### EXAMPLE 1

### Suppression of Neutrophil Extracellular Trap Formation by Exosomes from Poly IC activated Mesenchymal Stem Cells

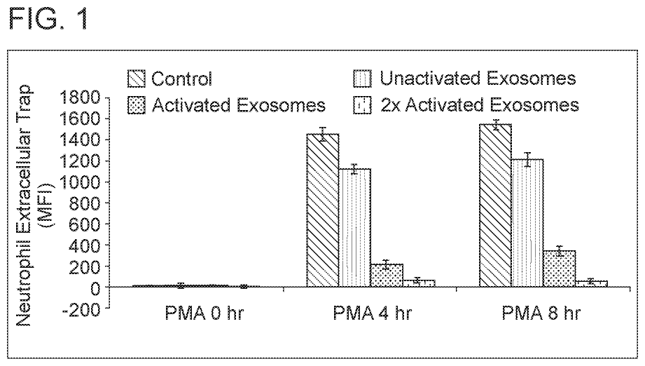

Umbilical cord mesenchymal stem cells were cultured in DMEM media with 10% fetal calf serum. Cells were activated with 100 ng/ml Poly IC for 2 hours. Exosomes were purified using Exo-quick kit according to the manufacturer's instructions. Exosomes where added at a concentration of 10 ng/ml based on protein content to neutrophils activated with PMA at 100 uM for the indicated timepoints. Neutrophil extracellular trap quantification was performed using the SYTOX method and quantified on flow cytometry as MFI. Results are shown in FIG. 1.

### EXAMPLE 2

### Exosomal Inhibition of Lung Pathology

A COVID-like lung injury was induced by administration of LPS. Addition of exosomes collected from activated mesenchymal stem cells, as described in Example 1, resulted in suppression of fluid leakage in the treated animals.

### REFERENCES

1. Brinkmann, V., et al., Neutrophil extracellular traps kill bacteria. Science, 2004.303(5663): p. 1532-5.
2. Zhu, Y., et al., Comprehensive characterization of neutrophil genome topology. Genes Dev, 2017. 31(2): p. 141-153.
3. Dumler, J. S., et al., Genome-Wide Anaplasma phagocytophilum AnkA-DNA Interactions Are Enriched in Intergenic Regions and Gene Promoters and Correlate with Infection-Induced Differential Gene Expression. Front Cell Infect Microbiol, 2016. 6: p. 97.
4. Lukasova, E., et al., Granulocyte maturation determines ability to release chromatin NETs and loss of DNA damage response; these properties are absent in immature AML granulocytes. Biochim Biophys Acta, 2013. 1833(3): p. 767-79.
5. Smetana, K., D. Mikulenkova, and H. Klamova, Heterochromatin density (condensation) during cell differentiation and maturation using the human granulocytic lineage of chronic myeloid leukaemia as a convenient model. Folia Biol (Praha), 2011. 57(5): p. 216-21.
6. Olins, A. L., et al., The human granulocyte nucleus: Unusual nuclear envelope and heterochromatin composition. Eur J Cell Biol, 2008. 87(5): p. 279-90.
7. Grigoryev, S. A. and C. L. Woodcock, Chromatin structure in granulocytes. A link between tight compaction and accumulation of a heterochromatin-associated protein (MENT). J Biol Chem, 1998. 273(5): p. 3082-9.
8. Cooper, P. R., L. J. Palmer, and I.L. Chapple, Neutrophil extracellular traps as a new paradigm in innate immunity: friend or foe? Periodontol 2000, 2013. 63(1): p. 165-97.
9. Sur Chowdhury, C., et al., Enhanced neutrophil extracellular trap generation in rheumatoid arthritis: analysis of underlying signal transduction pathways and potential diagnostic utility. Arthritis Res Ther, 2014. 16(3): p. R122.
10. Leppkes, M., et al., Externalized decondensed neutrophil chromatin occludes pancreatic ducts and drives pancreatitis. Nat Commun, 2016. 7: p. 10973.
11. Ghari, F., et al., Citrullination-acetylation interplay guides E2F-1 activity during the inflammatory response. Sci Adv, 2016. 2(2): p. e1501257.
12. Vollger, L., et al., Iron-chelating agent desferrioxamine stimulates formation of neutrophil extracellular traps (NETs) in human blood-derived neutrophils. Biosci Rep, 2016. 36(3).
13. Yost, C. C., et al., Neonatal NET-inhibitory factor and related peptides inhibit neutrophil extracellular trap formation. J Clin Invest, 2016. 126(10): p. 3783-3798.
14. Zepeda-Mendoza, C. J., et al., Phenotypic interpretation of complex chromosomal rearrangements informed by nucleotide-level resolution and structural organization of chromatin. Eur J Hum Genet, 2018. 26(3): p. 374-381.
15. Agraz-Cibrian, J. M., D. M. Giraldo, and S. Urcuqui-Inchima, 1,25-Dihydroxyvitamin D3 induces formation of neutrophil extracellular trap-like structures and modulates the transcription of genes whose products are neutrophil extracellular trap-associated proteins: A pilot study. Steroids, 2019. 141: p. 14-22.
16. Gosswein, S., et al., Citrullination Licenses Calpain to Decondense Nuclei in Neutrophil Extracellular Trap Formation. Front Immunol, 2019. 10: p. 2481.
17. Beato, M. and P. Sharma, Peptidyl Arginine Deiminase 2 (PADI2)-Mediated Arginine Citrullination Modulates Transcription in Cancer. Int J Mol Sci, 2020. 21(4).
18. Shi, L., et al., Endogenous PAD4 in Breast Cancer Cells Mediates Cancer Extracellular Chromatin Network Formation and Promotes Lung Metastasis. Mol Cancer Res, 2020. 18(5): p. 735-747.
19. Okochi, Y. and Y. Okamura, Regulation of Neutrophil Functions by Hv1/VSOP Voltage-Gated Proton Channels. Int J Mol Sci, 2021. 22(5).
20. Serov, D., et al., Calcium activity in response to nAChR ligands in murine bone marrow granulocytes with different Gr-1 expression. Cell Biol Int, 2021. 45(7): p. 1533-1545.
21. Zhai, T. Y., et al., Exosomes Released from CaSR-Stimulated PMNs Reduce Ischaemia/Reperfusion Injury. Oxid Med Cell Longev, 2021. 2021: p. 3010548.
22. Singh, P. K., et al., Pseudomonas aeruginosa quorum-sensing molecule N-(3-oxo-dodecanoyl)-L-homoserine lactone triggers mitochondrial dysfunction and apoptosis in neutrophils through calcium signaling. Med Microbiol Immunol, 2019. 208(6): p. 855-868.
23. Gorudko, I. V., et al., Neutrophil activation in response to monomeric myeloperoxidase. Biochem Cell Biol, 2018. 96(5): p. 592-601.
24. Chen, C. Y., et al., Anti-inflammatory effects of Perilla frutescens in activated human neutrophils through two independent pathways: Src family kinases and Calcium. Sci Rep, 2015. 5: p. 18204.
25. Herrmann, J. M. and J. Meyle, Neutrophil activation and periodontal tissue injury. Periodontol 2000, 2015. 69(1): p. 111-27.
26. Conejeros, I., et al., Effect of the synthetic Toll-like receptor ligands LPS, Pam3CSK4, HKLM and FSL-1 in the function of bovine polymorphonuclear neutrophils. Dev Comp Immunol, 2015. 52(2): p. 215-25.
27. Hallett, M. B., M. Al-Jumaa, and S. Dewitt, Optical methods for the measurement and manipulation of cytosolic calcium signals in neutrophils. Methods Mol Biol, 2014. 1124:p. 107-20.
28. Zhang, H., et al., STIM1 calcium sensor is required for activation of the phagocyte oxidase during inflammation and host defense. Blood, 2014. 123(14): p. 2238-49.
29. Prufer, S., et al., Distinct signaling cascades of TREM-1, TLR and NLR in neutrophils and monocytic cells. J Innate Immun, 2014. 6(3): p. 339-52.
30. Kappala, S. S., et al., FMLP-, thapsigargin-, and H(2)O(2)-evoked changes in intracellular free calcium concentration in lymphocytes and neutrophils of type 2 diabetic patients. Mol Cell Biochem, 2014. 387(1-2): p. 251-60.
31. Barnes, B. J., et al., Targeting potential drivers of COVID-19: Neutrophil extracellular traps. J Exp Med, 2020. 217(6).
32. Wei, M., et al., Inhibition of Peptidyl Arginine Deiminase 4-Dependent Neutrophil Extracellular Trap Formation Reduces Angiotensin II-Induced Abdominal Aortic Aneurysm Rupture in Mice. Front Cardiovasc Med, 2021. 8: p. 676612.
33. Vitiello, A., et al., Pharmacological approach for the reduction of inflammatory and prothrombotic hyperactive state in COVID-19 positive patients by acting on complement cascade. Hum Immunol, 2021. 82(4): p. 264-269.
34. Ng, N. and C. A. Powell, Targeting the Complement Cascade in the Pathophysiology of COVID-19 Disease. J Clin Med, 2021. 10(10).
35. Fodil, S. and D. Annane, Complement Inhibition and COVID-19: The Story so Far. Immunotargets Ther, 2021. 10: p. 273-284.
36. Araten, D. J., et al., Mild Clinical Course of COVID-19 in 3 Patients Receiving Therapeutic Monoclonal Antibodies Targeting C5 Complement for Hematologic Disorders. Am J Case Rep, 2020. 21: p. e927418.
37. Pike, A., et al., COVID-19 infection in patients on anti-complement therapy: The Leeds National Paroxysmal Nocturnal Haemoglobinuria service experience. Br J Haematol, 2020. 191(1): p. e1-e4.
38. McEneny-King, A. C., et al., Pharmacokinetic and Pharmacodynamic Evaluation of Ravulizumab in Adults with Severe Coronavirus Disease 2019. Infect Dis Ther, 2021. 10(2): p. 1045-1054.
39. Kulkarni, S., et al., Repurposed immunomodulatory drugs for Covid-19 in pre-ICu patient—mulTi-Arm Therapeutic study in pre-ICu patients admitted with Covid-19—Repurposed Drugs (TACTIC-R): A structured summary of a study protocol for a randomised controlled trial. Trials, 2020. 21(1): p. 626.
40. Smith, K., et al., A Phase 3 Open-label, Randomized, Controlled Study to Evaluate the Efficacy and Safety of Intravenously Administered Ravulizumab Compared with Best Supportive Care in Patients with COVID-19 Severe Pneumonia, Acute Lung Injury, or Acute Respiratory Distress Syndrome: A structured summary of a study protocol for a randomised controlled trial. Trials, 2020. 21(1): p. 639.
41. Iluta, S., et al., Haematology patients infected with SARS-CoV-2, pretreated with eculizumab or siltuximab, develop oligosymptomatic disease. Eur J Hosp Pharm, 2021.
42. Tatar, E., et al., Importance of eculizumab treatment in recurrence of atypical hemolytic uremic syndrome during the SARS-CoV-2 pandemic. Int Urol Nephrol, 2021.
43. Hofstadt-van Oy, U., et al., Complement inhibition initiated recovery of a severe myasthenic crisis with COVID-19. J Neurol, 2021. 268(9): p. 3125-3128.
44. Boudhabhay, I., et al., Case Report: Adult Post-COVID-19 Multisystem Inflammatory Syndrome and Thrombotic Microangiopathy. Front Immunol, 2021. 12: p. 680567.
45. Rodriguez-Guerra, M., P. Jadhav, and T. J. Vittorio, Current treatment in COVID-19 disease: a rapid review. Drugs Context, 2021. 10.
46. Tiede, A., et al., Prothrombotic immune thrombocytopenia after COVID-19 vaccination. Blood, 2021. 138(4): p. 350-353.
47. Parackova, Z., et al., Neutrophil Extracellular Trap Induced Dendritic Cell Activation Leads to Th1 Polarization in Type 1 Diabetes. Front Immunol, 2020. 11: p. 661.
48. Weerappuli, P. D., et al., Extracellular Trap-Mimicking DNA-Histone Mesostructures Synergistically Activate Dendritic Cells. Adv Healthc Mater, 2019. 8(22): p. e1900926.
49. Scozzi, D., et al., Neutrophil extracellular trap fragments stimulate innate immune responses that prevent lung transplant tolerance. Am J Transplant, 2019. 19(4): p. 1011-1023.
50. Qiu, S. L., et al., Neutrophil extracellular traps induced by cigarette smoke activate plasmacytoid dendritic cells. Thorax, 2017. 72(12): p. 1084-1093.
51. Kahlenberg, J. M., et al., Neutrophil extracellular trap-associated protein activation of the NLRP3 inflammasome is enhanced in lupus macrophages. J Immunol, 2013. 190(3): p. 1217-26.
52. Tillack, K., et al., T lymphocyte priming by neutrophil extracellular traps links innate and adaptive immune responses. J Immunol, 2012. 188(7): p. 3150-9.
53. Zhang, H., et al., Erythromycin suppresses neutrophil extracellular traps in smoking-related chronic pulmonary inflammation. Cell Death Dis, 2019. 10(9): p. 678.
54. Muller, S., et al., Dimethylfumarate Impairs Neutrophil Functions. J Invest Dermatol, 2016. 136(1): p. 117-26.
55. Yuan, B., et al., Bone marrow stromal cells induce an ALDH+ stem cell-like phenotype and enhance therapy resistance in AML through a TGF-beta-p38-ALDH2 pathway. PLoS One, 2020. 15(11): p. e0242809.
56. Potian, J. A., et al., Veto-like activity of mesenchymal stem cells: functional discrimination between cellular responses to alloantigens and recall antigens. J Immunol, 2003. 171(7):p. 3426-34.
57. Muscari, C., et al., Polyamine depletion reduces TNFalpha/MG132-induced apoptosis in bone marrow stromal cells. Stem Cells, 2005. 23(7): p. 983-91.
58. Petrini, M., et al., Identification and purification of mesodermal progenitor cells from human adult bone marrow. Stem Cells Dev, 2009. 18(6): p. 857-66.
59. Lasala, G. P. and J. J. Minguell, Bone marrow-derived stem/progenitor cells: their use in clinical studies for the treatment of myocardial infarction. Heart Lung Circ, 2009. 18(3):p. 171-80.
60. Trombi, L., et al., Selective culture of mesodermal progenitor cells. Stem Cells Dev, 2009. 18(8): p. 1227-34.
61. Li, M., et al., CXCR4 positive bone mesenchymal stem cells migrate to human endothelial cell stimulated by ox-LDL via SDF-1alpha/CXCR4 signaling axis. Exp Mol Pathol, 2010. 88(2): p. 250-5.
62. Balber, A. E., Concise review: aldehyde dehydrogenase bright stem and progenitor cell populations from normal tissues: characteristics, activities, and emerging uses in regenerative medicine. Stem Cells, 2011. 29(4): p. 570-5.
63. Bell, G. I., et al., Transplanted human bone marrow progenitor subtypes stimulate endogenous islet regeneration and revascularization. Stem Cells Dev, 2012. 21(1): p. 97-109.
64. Yang, H., et al., Protection of bone marrow mesenchymal stem cells from acute lung injury induced by paraquat poisoning. Clin Toxicol (Phila), 2011. 49(4): p. 298-302.
65. Tu, X. H., et al., Role of bone marrow-derived mesenchymal stem cells in a rat model of severe acute pancreatitis. World J Gastroenterol, 2012. 18(18): p. 2270-9.
66. Ren, M., et al., Ginkgo biloba L. extract enhances the effectiveness of syngeneic bone marrow mesenchymal stem cells in lowering blood glucose levels and reversing oxidative stress. Endocrine, 2013. 43(2): p. 360-9.
67. Shen, L. H., et al., Protective effects of MCI-186 on transplantation of bone marrow stromal cells in rat ischemic stroke model. Neuroscience, 2012. 223: p. 315-24.
68. Jin, G., et al., Allogeneic bone marrow-derived mesenchymal stem cells attenuate hepatic ischemia-reperfusion injury by suppressing oxidative stress and inhibiting apoptosis in rats. Int J Mol Med, 2013. 31(6): p. 1395-401.
69. Yang, H., et al., Combined treatment with bone marrow mesenchymal stem cells and methylprednisolone in paraquat-induced acute lung injury. BMC Emerg Med, 2013. 13 Suppl 1: p. S5.
70. Windmolders, S., et al., Mesenchymal stem cell secreted platelet derived growth factor exerts a pro-migratory effect on resident Cardiac Atrial appendage Stem Cells. J Mol Cell Cardiol, 2014. 66: p. 177-88.
71. Zhu, H., et al., Aldehyde dehydrogenase-2 is a host factor required for effective bone marrow mesenchymal stem cell therapy. Arterioscler Thromb Vasc Biol, 2014. 34(4): p. 894-901.
72. Wu, J., et al., TGF-beta1 induces senescence of bone marrow mesenchymal stem cells via increase of mitochondrial ROS production. BMC Dev Biol, 2014. 14: p. 21.
73. Xia, W., et al., Improved survival of mesenchymal stem cells by macrophage migration inhibitory factor. Mol Cell Biochem, 2015. 404(1-2): p. 11-24.
74. Ashour, R. H., et al., Comparative study of allogenic and xenogeneic mesenchymal stem cells on cisplatin-induced acute kidney injury in Sprague-Dawley rats. Stem Cell Res Ther, 2016. 7(1): p. 126.
75. Sherman, S. E., et al., High Aldehyde Dehydrogenase Activity Identifies a Subset of Human Mesenchymal Stromal Cells with Vascular Regenerative Potential. Stem Cells, 2017. 35(6): p. 1542-1553.
76. Zhang, Z. H., et al., Mesenchymal stem cells increase expression of heme oxygenase-1 leading to anti-inflammatory activity in treatment of acute liver failure. Stem Cell Res Ther, 2017. 8(1): p. 70.
77. Kalhori, Z., et al., Improvement of the folliculogenesis by transplantation of bone marrow mesenchymal stromal cells in mice with induced polycystic ovary syndrome. Cytotherapy, 2018. 20(12): p. 1445-1458.
78. Ma, Z., et al., Bone marrow-derived mesenchymal stromal cells ameliorate severe acute pancreatitis in rats via hemeoxygenase-1-mediated anti-oxidant and anti-inflammatory effects. Cytotherapy, 2019. 21(2): p. 162-174.
79. Zhang, F., et al., New strategy of bone marrow mesenchymal stem cells against oxidative stress injury via Nrf2 pathway: oxidative stress preconditioning. J Cell Biochem, 2019. 120(12): p. 19902-19914.
80. Zhang, F., et al., PARK7 enhances antioxidative-stress processes of BMSCs via the ERK1/2 pathway. J Cell Biochem, 2021. 122(2): p. 222-234.
81. Capoccia, B. J., et al., Revascularization of ischemic limbs after transplantation of human bone marrow cells with high aldehyde dehydrogenase activity. Blood, 2009. 113(21): p. 5340-51.
82. Keller, L. H., Bone marrow-derived aldehyde dehydrogenase-bright stem and progenitor cells for ischemic repair. Congest Heart Fail, 2009. 15(4): p. 202-6.
83. Najar, M., et al., Isolation and Characterization of Bone Marrow Mesenchymal Stromal Cell Subsets in Culture Based on Aldehyde Dehydrogenase Activity. Tissue Eng Part C Methods, 2018. 24(2): p. 89-98.
84. Zhang, W., et al., [rotective effect of bone marrow mesenchymal stem cells-derived exosomes against testicular ischemia-reperfusion injury in rats]. Nan Fang Yi Ke Da Xue Xue Bao, 2018. 38(8): p. 910-916.
85. Li, Y., et al., Effects of Sca-1(+) bone marrow mesenchymal stem cells on lung ischemia-reperfusion injury. J Biol Regul Homeost Agents, 2019. 33(3): p. 745-752.
86. Demirayak, B., et al., Effect of bone marrow and adipose tissue-derived mesenchymal stem cells on the natural course of corneal scarring after penetrating injury. Exp Eye Res, 2016. 151: p. 227-35.
87. Choi, D. H., et al., Tonsil-derived mesenchymal stem cells incorporated in reactive oxygen species-releasing hydrogel promote bone formation by increasing the translocation of cell surface GRP78. Biomaterials, 2021. 278: p. 121156.
88. Hoffman, A. M., et al., Lung-derived mesenchymal stromal cell post-transplantation survival, persistence, paracrine expression, and repair of elastase-injured lung. Stem Cells Dev, 2011. 20(10): p. 1779-92.
89. Roehrich, M. E., et al., Characterization of cardiac-resident progenitor cells expressing high aldehyde dehydrogenase activity. Biomed Res Int, 2013. 2013: p. 503047.
90. Wang, L., et al., Preincubation with a low-dose hydrogen peroxide enhances anti-oxidative stress ability of BMSCs. J Orthop Surg Res, 2020. 15(1): p. 392.

1. A method of reducing neutrophil secretion of neutrophil extracellular traps comprising administering a mesenchymal stem cell and/or products derived from said mesenchymal stem cells to a patient in need of treatment.

2. The method of claim 1, wherein said mesenchymal stem cells are activated in a manner to enhance ability to inhibit neutrophil production of NETs by direct contact or by secretion of soluble factors.

3. The method of claim 1, wherein said NETs are comprised of DNA and histones.

4. The method of claim 1, wherein said mesenchymal stem cells are autologous to the host.

5. The method of claim 1, wherein said mesenchymal stem cells are allogeneic to the host.

6. The method of claim 1, wherein said mesenchymal stem cells are xenogeneic to the host.

7. The method of claim 1, wherein said mesenchymal stem cells are derived from a tissue selected from the group consisting of: a) bone marrow; b) liver; c) spleen; d) adipose tissue; e) peripheral blood; f) mobilized peripheral blood; g) cerebral spinal fluid; h) menstrual blood; i) tonsils; j) deciduous tooth; k) fallopian tube; l) endometrium; m) muscle; and n) hair follicle.

8. The method of claim 7, wherein said mesenchymal stem cells express SCA-1.

9. The method of claim 7, wherein said mesenchymal stem cells express interleukin-1 receptor.

10. The method of claim 7, wherein said mesenchymal stem cells express interleukin-3 receptor.

11. The method of claim 7, wherein said mesenchymal stem cells express interleukin-6 receptor.

12. The method of claim 7, wherein said mesenchymal stem cells express interleukin-10 receptor.

13. The method of claim 7, wherein said mesenchymal stem cells express leukemia inhibitor factor receptor.

14. The method of claim 7, wherein said mesenchymal stem cells express HGF-1 receptor.

15. The method of claim 7, wherein said mesenchymal stem cells express VEGF receptor.

16. The method of claim 7, wherein said mesenchymal stem cells express CD133.

17. The method of claim 7, wherein said me8senchymal stem cells express CD90.

18. The method of claim 7, wherein said mesenchymal stem cells express PD-L1.

19. The method of claim 7, wherein said mesenchymal stem cells express CTLA-4.

20. The method of claim 7, wherein said mesenchymal stem cells express FoxP3.
